(19) United States
(12) Patent Application Publication
(10) Pub. No.: US 2012/0058962 A1
(43) Pub. Date: Mar. 8, 2012

(54) BUCCAL AND/OR SUBLINGUAL THERAPEUTIC FORMULATION

(75) Inventors: Alistair Cumming, Mornington Vic (AU); Lance Sparrow, Glen Iris Vic (AU); David Kannar, Mt Eliza Vic (AU)

(73) Assignee: Lingual Conseqna Pty Ltd., Collington, Victoria (AU)

(21) Appl. No.: 13/256,844

(22) PCT Filed: May 20, 2010
(86) PCT No.: PCT/AU2010/000594

§ 371 Date: Sep. 15, 2011

(30) Foreign Application Priority Data
May 20, 2009 (AU) 2009902280

(51) Int. Cl. A61K 31/704; A61K 31/137; A61K 31/4045; A61K 47/10; A61K 31/495; A61K 47/08; A61K 47/36; A61K 47/40; A61K 31/192; A61K 31/7008

(52) U.S. Cl. 514/26; 514/569; 514/570; 514/653; 514/419; 514/62; 514/255.04; 514/772; 514/777

(57) ABSTRACT

A buccal and/or sublingual formulation comprising one or more active compounds; and a buccal matrix which releases the active compounds at a predetermined rate for transport across the buccal and/or sublingual membranes, wherein the rate of release of the active compounds is either (A) the same or substantially the same rate at which the active compounds are transported across the buccal and/or sublingual membranes; or (B) a rate which releases the active compounds over an extended period as required by the therapeutic affect or treatment window for those active compounds.

### FIELD OF THE INVENTION

The invention relates to a delivery system which provides improved delivery of therapeutic compounds. In particular, the present invention relates to buccal and sublingual formulations.

### BACKGROUND OF THE INVENTION

In this specification, where a document, act or item of knowledge is referred to or discussed, this reference or discussion is not an admission that the document, act or item of knowledge or any combination thereof was at the priority date, publicly available, known to the public, part of common general knowledge; or known to be relevant to an attempt to solve any problem with which this specification is concerned.

It is known that the action of a therapeutic compound can be modified using specific excipients in the delivery formulation. In addition, the formulation itself is often critical to the efficacy of the compound to be delivered. One class of agents which has been used for this purpose is the polyethylene glycols (PEGs). An example of disclosure of a formulation using PEGs in this manner is international patent application no WO 2006/105615. However, known formulations using PEGs to date have not provided optimum control of the active compound release rate to provide a range of onsets of action (ie, from slow to rapid).

The ability to effectively deliver therapeutic compounds to animals and, in particular, humans is frequently dependent on compliance of the recipient. Poor patient compliance is a significant barrier to the completion of prescription regimens and the cause of sub-optimal clinical outcomes. Compliance is also often connected to or associated with the formulation used to deliver the compound. It is known that many orally delivered active compounds also deliver either an unsatisfactory taste in the mouth or generate burning in the throat. For these reasons, such compounds presently have to be swallowed prior to breakdown of the matrix and release of the active. Managing problematic taste and other sensations are thus important for patient compliance.

Accordingly, in addition to the need to be able to control the release rate, the buccal and/or sublingual delivery of many of the current commercially available oral active compounds has not been pursued because of their offensive or unpalatable taste, unpleasant mouth feel due to chalkiness, grittiness, dryness or astringency, low solubility in saliva or poor bioavailability.

There is a continual need to develop more improved drug delivery formulations which:
efficaciously deliver therapeutic agents quickly yet without inducing unwanted side effects; and/or
reduce the side-effects that impact on patient compliance; and/or
provide improved control of the release rate within a range (from slow to rapid) of onsets of action, by using a variety of enhancers and complexing agents (individually or in combination) to provide that tighter control.

### SUMMARY OF THE INVENTION

It has been found that a composition comprising at least one active compound with selected excipients, complexing agents, and/or carriers can provide improved solubility and permeability to improve the release kinetics of the active compound(s) (when delivered either sublingually or buccally) and increase delivery of the active compound(s). This results in more reproducible plasma profiles and a better managed onset of clinical effect by reason of higher bioactivity, that is, an improved pharmacokinetic profile for the active compound as measured by standard testing parameters (eg: Tₘₐₓ, Cₘₐₓ and AUC (“area under the curve”, a measure of drug concentration) values in their known forms).

The term “buccal and/or sublingual formulation” as used herein refers to a drug delivery formulation wherein an active compound is provided for absorption across one or more membranes in the buccal cavity, including the buccal mucosa, buccal gingiva, mucous membrane of the tongue, sublingual membrane and the soft palate. The term encompasses all suitable solid and semi-solid dosage forms, including troches, sublingual tablets, and buccal tablets (i.e. a preparation which can be placed under the tongue). The term “buccal” is used in its broadest sense to refer to the oral cavity as a whole.

The present invention is expected to provide a tailored matrix which is capable of being modified to either:
release an active compound(s) at the same, or substantially the same, rate at which the active compound(s) are transported across the buccal and/or sublingual membranes to ensure the rapid balanced transport into the bloodstream and thus deliver higher bioactivity; or
release an active compound(s) over an extended period for those active compounds which require longer therapeutic affect windows (or extended AUC's). (Without being bound by theory, the active compound(s) may also be transported into the buccal or sublingual membrane to be released over an extended period of time, ie the membrane acts as a “reservoir”.)

According to a first aspect of the invention, there is provided a buccal and/or sublingual formulation comprising:
(a) one or more active compounds; and
(b) a matrix which releases the active compounds at a predetermined rate for transport across the buccal and/or sublingual membranes, the matrix comprising one or more compounds selected predominantly from the group consisting of:
   (i) taste masking agents,
   (ii) enhancers,
   (iii) complexing agents, and
   mixtures thereof; and
(c) other pharmaceutically acceptable carriers and/or excipients,
   wherein the rate of release of the active compounds is either (A) the same or substantially the same rate at which the active compounds are transported across the buccal and/or sublingual membranes; or (B) a rate which releases the active compounds so as to provide a higher area under the curve (AUC) value when compared with equivalent compounds in a swallow formulation on a dose normalised basis.

A person skilled in the art will understand that the transport in (A) above can be either passive transport or active transport assisted by means of the influence of an agent such as a permeation enhancer. This rate of transport in (A) can also be further increased using a combination of effects delivered by different excipients within the matrix. For example:
changing the pH will improve solubility of some salts.
increasing the rate of disintegration of the matrix will release more active more quickly at the membrane interface.
if the transport rate of the active is slower than the release rate, then it is important to also use a permeation enhancer to increase the rate at which the active compound passes through the membrane.

A person skilled in the art will understand that the higher AUC in (B) above can be achieved in different ways using a combination of effects delivered by different excipients within the matrix. For example,
an earlier onset of action (when compared with a typical swallow formulation) can be achieved by increasing membrane permeability and thus facilitating an even faster uptake of the active;
releasing the active compound over an extended period can be achieved by complexing the active compound to retard release as required by the therapeutic affect window for that active compound.

It will be appreciated by those skilled in the art that a particular excipient may perform more than one function. For example, an enhancer may facilitate a higher uptake rate and also provide a taste masking effect or a sweetener/flavour may improve palatability and act to reduce throat catch.

A person skilled in the art will understand that the selection of appropriate active compounds (such as specific salts or derivatives thereof) for use in a formulation according to the invention can partly alleviate solubility issues. A person skilled in the art will also understand that the “equivalent compounds in a swallow formulation” in (B) above refers to compounds having the same active core as the active compounds in the formulation according to the invention, however the active compounds used in a formulation according to the invention may be a different salt or derivative thereof.

Additionally, it is important to understand that the active compounds must then be matched with a range of enhancers to provide the predetermined release rate, in addition to taste masking agents to negate taste issues. When matched appropriately, the predetermined Tₘₐₓ, Cₘₐₓ and AUC may be achieved.

Reference herein to an “active compound” or “biologically active compound” includes a therapeutic or prophylactic agent, drug, pro-drug, drug complex, drug intermediate, diagnostic agent, enzyme, medicine, plant extract, herbal extract, infusion or concoction, phytochemical, protein, antibody, antibody fragment or derivative, bioactive compound or dietary supplement.

The term “matrix” as used herein refers to a solid or semi-solid monolithic material containing one or more dissolved or dispersed active compounds closely associated with a surrounding, rate-controlling heterogenous material where the active compound or compounds exhibit a zero- or first-order release rate when the matrix is placed in direct contact with a moist diffusion membrane. The solid or semi solid monolithic material can include a range of materials known in the art of pharmaceutical drug delivery to taste mask, emulsify, solubilize, complex or enhance delivery of any biologically active lipophilic or hydrophilic compound across a membrane.

The term “taste masking agents” when used herein refers to taste receptor blockers, compounds which mask the chalkiness, grittiness, dryness and/or astringent taste properties of an active compound, compounds which reduce throat catch as well as compounds which add a flavour. The following are examples:
Taste receptor blockers include Kyron T-134, a glycoprotein extract called miraculin from the fruit of the plant synsepalum dulcificum, ethyl cellulose, hydroxypropyl methylcellulose, arginine, sodium carbonate, sodium bicarbonate, gustducin blockers and mixtures thereof
Compounds which mask the chalkiness, grittiness, dryness and/or astringent taste properties of an active compound include those of a natural or synthetic fatty type or other flavorant such as cocoa, chocolate (especially mint chocolate), cocoa butter, milk fractions, vanillin butter fat, egg or egg white, peppermint oil, wintergreen oil, spearmint oil and similar oils.
Compounds which reduce throat catch include combinations of high and low solubility acids. For example high solubility acids suitable for use here include amino acids (eg alanine, arginine etc), glutaric, ascorbic, malic, oxalic, tartaric, malonic, acetic, citric acids and mixtures thereof. Low solubility acids suitable for use include oleic, stearic and aspartic acids plus certain amino acids such as glutamic acid, glutamine, histidine, isoleucine, leucine, methionine, phenylalanine, serine, tryptophan, tyrosine, valine and fumaric acid. Actual amounts used will vary depending on the amount of throat catch or burn exhibited by the active used but will generally be in the range of 1-40%.
Flavouring agents include sweeteners and flavours. Examples of suitable sweeteners and flavours include mannitol, sorbitol, maltitol, lactitol, isomaltitol, erythritol, xylitol, sucrose, ammonium glycyrrhizinate, mango aroma, black cherry aroma, sodium citrate, colloidal silicium dioxide, sucralose; zinc gluconate; ethyl maltitol; glycine; acesulfame-K; aspartame; saccharin; acesulfam K, neohesperidin DC, thaumatin, stevioside, fructose; xylitol; honey; honey extracts; corn syrup, golden syrup, misri, spray dried licorice root; glycerrhizine; dextrose; sodium gluconate; stevia powder; glucono delta-lactone; ethyl vanillin; vanillin; normal and high-potency sweeteners or syrups or salts thereof and mixtures thereof. Other examples of appropriate flavouring agents include coffee extract, mint; lamiacea extracts; citrus extracts; almond oil; babassu oil; borage oil; blackcurrant seed oil; canola oil; castor oil; coconut oil; corn oil; cottonseed oil; evening primrose oil; grapeseed oil; groundnut oil; mustard seed oil; olive oil; palm oil; palm kernel oil; peanut oil; grapeseed oil; safflower oil; sesame oil; shark liver oil; soybean oil; sunflower oil; hydrogenated castor oil; hydrogenated coconut oil; hydrogenated palm oil; hydrogenated soybean oil; hydrogenated vegetable oil; hydrogenated cottonseed and castor oil; partially hydrogenated soybean oil; soy oil; glyceryl tricaproate; glyceryl tricaprylate; glyceryl tricaprate; glyceryl triundecanoate; glyceryl trilaurate; glyceryl trioleate; glyceryl trilinoleate; glyceryl trilinolenate; glyceryl tricaprylate/caprate; glyceryl tricaprylate/caprate/laurate; glyceryl tricaprylate/caprate/linoleate; glyceryl tricaprylate/caprate/stearate; saturated polyglycolized glycerides; linoleic glycerides; caprylic/capric glycerides; modified triglycerides; fractionated triglycerides; safrole, citric acid, d-limonene, malic acid and phosphoric acid or salts and/or mixtures thereof.

The term “enhancers” when used herein refers to agents which work to increase membrane permeability and/or work to increase the solubility of a particular active. Both issues can be pivotal to the properties of the formulation. The following are examples.
Chelators: EDTA, citric acid, sodium salicylate, methoxysalicylates. (See Senel & Hincal: JCR 72 2001 133-144; Malhalingam et al: AAPS Pharmascitech 2007 (8) vol 3 Article 55).
Surfactants: sodium lauryl sulphate, polyoxyethylene, POE-9-laurylether, POE-20-cetylether, benzalkonium chloride, 23-lauryl ether, cetylpyridinium chloride, cetyltrimethyl ammonium bromide, amphoteric and cationic surfactants.
Membrane disrupting compounds such as powdered alcohols (eg menthol and ethanol), and compounds such as lipophilic enhancers which are safe to be used orally. (Nicolazzo, Reid and Finnin J Pharmaceutical Sciences Vol 93, No 8 Aug. 2004 2054-2063).
Fatty and other acids: oleic acid, capric acid, lauric acid, lauric acid/propylene glycol, methyloleate, ysophosphatidylcholine, phosphatidylcholine (Sudhakar et al JCR 114 (2006) 15-40), oleic acid co-delivered with PEG 200, (Lee and Kellaway Int J Pharmaceutics 204 (2000) 137-144).
Lysalbinic acid (Starokadomdkyy & Dubey Int J Pharmaceutics 308 (2006) 149-154).
Non-surfactants such as unsaturated cyclic ureas.
Others: glucosaminoglycans (GAGs), aprotinin, azone, cyclodextrin, dextran sulfate, curcumin, menthol, polysorbate 80, sulfoxides and various alkyl glycosides.
Chitosan-4-thiobutylamide, chitosan-4-thiobutylamide/GSH, chitosan-cysteine, chitosan-(85% degree N-deacetylation), poly(acrylic acid)-homocysteine, polycarbophil-cysteine, polycarbophil-cysteine/GSH, chitosan-4-thioethylamide/GSH, chitosan-4-thioglycholic acid. Hyaluronic acid in 3 MW's (Sandri et al: J Pharmacy and Pharmacology 2004, 56: 1083-1090.)
Bile Salts (Dihydroxy and Trihydroxy), sodium glycocholate, sodium deoxycholate, sodium taurocholate, sodium glycodeoxycholate, sodium taurodeoxycholate(Artusi et al: Int J Pharmaceutics 250 (2003) 203-213).
Propanolol hydrochloride (Akbari et al: Il Farmaco 59 (2004)155-161).

The selection of the glucoaminoglycans (GAGs) and the amount used will depend on the active compound(s) to be included in the formulation. A person skilled in the art will be able to select a suitable GAG to achieve the predetermined pharmacokinetics for a particular active ingredient because the properties of GAGs are well known. For example, GAGs such as chitosan and hyaluronic acid exhibit a higher swelling profile and slower erosion rate producing sustained release characteristics. It is known in public art that GAGs have the ability to influence bioequivalence. —Mar. Drugs 2010, 8: 1305-1322[17]. The term “complexing agents” when used herein includes agents in the group consisting of:
Cyclodextrins. Cyclodextrins are obtained from the enzymatic hydrolysis of starch and, depending on the enzyme used, the Alpha (6 glucose units), Beta (7 glucose units) or Gamma (8 glucose units) forms are obtained, which differ in the diameter of the circle and, therefore, may form complexes with products having a higher or lower molecular weight. The most widely used is beta-cyclodextrin, which is composed of 7 glucose units cyclically bonded to form a ring. When these complexes are formed, the functional group responsible for a product's bad taste may become “blocked” by the new bonds formed.
There are other compounds in the market with a high number of hydroxyl groups that are used in pharmaceutical processes, such as other carbohydrates like glucose, mannose or galactose, or polyalcohols derived from these carbohydrates, such as mannitol or sorbitol. The most widely known application of these polyalcohols, more specifically, of mannitol and sorbitol, in pharmacy is mainly as diluents in pharmaceutical forms in powder or tablets, both for humid granulation of the mixtures and for direct compression. They are very widely used in the manufacturing of sugar-substitute chewable tablets, since they are not cariogenic and they provide fewer calories to the final product. Mannitol and sorbitol may also be used as plasticisers for the gelatin used in soft-gelatin capsules adapted to contain active principles; and also as crystallisation inhibitors in sugar syrups. In addition, mannitol is also used as a lyophilisation excipient because it favours the sublimation process. Many of these compounds have the advantage of also being taste masking agents.
Buffering materials can be both used to increase solubility and enhance adsorption of active compounds. Examples of suitable buffering materials or antacids suitable for use herein comprise any relatively water-soluble antacid acceptable to the Food & Drug Administration, such as aluminum carbonate, aluminum hydroxide (or as aluminum hydroxide-hexitol stabilized polymer, aluminum hydroxide-magnesium hydroxide co-dried gel, aluminum hydroxide-magnesium trisilicate codried gel, aluminum hydroxide-sucrose powder hydrated), aluminum phosphate, aluminum hydroxy carbonate, dihydroxyaluminum sodium carbonate, aluminum magnesium glycinate, dihydroxyaluminum aminoacetate, dihydroxyaluminum aminoacetic acid, bismuth aluminate, bismuth carbonate, bismuth subcarbonate, bismuth subgallate, bismuth subnitrate, calcium carbonate, calcium phosphate, hydrated magnesium aluminate activated sulfate, magnesium aluminate, magnesium aluminosilicates, magnesium carbonate, magnesium glycinate, magnesium hydroxide, magnesium oxide and magnesium trisilicate, and or mixtures thereof. Preferred buffering materials or antacids include aluminum hydroxide, calcium carbonate, magnesium carbonate and mixtures thereof, as well as magnesium hydroxide. Many of these compounds have the advantage of also being taste masking agents particularly useful for addressing throat catch.
the group consisting of amphoteric surfactants, cationic surfactants, amino acids having nitrogen functional groups and proteins rich in these amino acids.

A person skilled in the art would understand that the buffering agents are modifying the pH of the formulation to minimise damage to the mucosal membranes, for example, by an acidic active compound.

Preferred complexing or enhancing agents include PEGs, chitosan, hyaluronic acid, cyclodextrins and polyalcohols. It should be noted that preference for a complexing agent is primarily governed by the specific requirements of the active to be delivered.

The selection of the other excipients, such as permeation enhancers, disintegrants, masking agents, binders, flavours, sweeteners and taste maskers, is specifically matched to the active depending on the predetermined pharmacokinetic profile and/or organoleptic outcome.

A person skilled in the art will understand that the term “active compounds” includes approved pharmaceutical ingredients (API).

The invention relates to a formulation which can be used with a wide range of active compounds and combinations of active compounds. Whilst each active ingredient will have its own characteristics, these characteristics will be known to the person skilled in the art and that person will be able to easily develop a formulation according to the invention. Further, it is common for some active ingredients to be administered together as they have a complementary or synergistic effect.

Examples of suitable active compounds include but are not limited to anti-infective agents (antibiotics), eye, ear, nose and throat preparations, anti-neoplastic agents including antibody, nanobody, antibody fragment(s), antibody directed enzyme pro-drug therapy (ADEPT), gastrointestinal drugs, respiratory agents, arthritic agents, antihistamines, anti-emetics, blood formation and coagulation agents, diagnostic agents, hormones and synthetic substitutes, cardiovascular drugs, (including but not limited to fibrinolytics, hypocholesterolaemic and hyperlipidaemia agents, platelet thinning agents), hypothyroidism drugs, psychoactive drugs, immunotherapy agents, skin and mucous membrane preparations, NSAIDs, analgesics, anaesthetics (including but not limited to pre-anaesthetics and post-analgesics especially where nausea and vomiting limit oral administration), muscle spasm medications, anti-inflammatory agents, central nervous system drugs, dietary supplements, plant extracts, photosensitizing agents, hyposensitizing agents, heterodimers, monomers, oligomers, homodimers, diabetic agents, and electrolyte and water balance agents as single actives, salts, mixtures, pain relief agents, ibuprofen, ketaprofen, acetaminophen/paracetamol, diclofenac, opoids, proteins, peptides, pro-drugs, drug complexes, drug intermediates, vitamins and minerals, derivatives, enzyme or protein and protein complexes including but not limited to vaccines.

Other active compounds include for example a bisphosphonic acid or a bisphosphonate salt, CoQ10, immunotherapy and anti-allergy agents, hormones of natural or synthetic (also known as bioidentical) origin, insulin, triamcinolone, testosterone, levonorgestrel, estradiol, phytoestrogen, estrone, dexamethasone, ethynodiol, prednisone, desogestrel, cyproterone, norethindrone, megestrol, hydrocortisone, danazol, cetirizine, levocetirizine dihydrochloride, statins, cox-2 inhibitors, expectorants, dextromethorphan, cortisone acetate, aviane, nandrolone, fluoxymesterone, fludrocortisone, fluoxymesterone dexamethasone, levora fludrocortisone, low-ogestrel methylprednisolone, necon, estropipate, levoxyl, methimazole, propylthiouracil desmopressin, zolpidem, pentosan polysulfate, progesterone, prednisolone, orgestrel, trivora, venlafaxine, hydrochloride, zovia, black elder berry extracts (sambucus nigra), gestodene, alfacalcidol, 1,25-dihydroxyvitamin D3, clomiphene, finasteride and tibolone or any biologically relevant intermediate or a combination of two or more of any of the above-mentioned agents especially where vomiting, nausea or other clinical parameters limit oral administration. Preferred bisphosphonic acids or bisphonate salts are selected from the group comprising alendronate, etidronate, pamidronate, tiludronate, risedronate and alendronate compounds. Even more preferably, the bisphosphonic acid is alendronate selected from the group comprising anhydrous alendronate or hydrated alendronate salts, such as sodium alendronate.

The formulation also includes other pharmaceutically acceptable carriers and/or excipients such as binders, lubricants, diluents, coatings, disintegrants, barrier layer components, glidants, colouring agents, solubility enhancers, gelling agents, fillers, proteins, co-factors, emulsifiers, solubilising agents, suspending agents and mixtures thereof.

A person skilled in the art would know what other pharmaceutically acceptable carriers and/or excipients could be included in the formulations according to the invention. The choice of excipients would depend on the characteristics of the compositions and on the nature of other pharmacologically active compounds in the formulation. Appropriate excipients are known to those skilled in the art (see Handbook Of Pharmaceutical Excipients, fifth edition, 2005 edited by Rowe et al., McGraw Hill). For example Maize starch might act as a binder, a diluent and as a disintegrating agent.

Examples of appropriate other excipients include:
suspending agents to improve texture and consistency selected from the group consisting of tetragonolobus, Acacia glaucophylla, Acacia abyssinica, Acacia nilotica, Acacia gummifera, Acacia arabica, silica gel, kollidon, cremaphor, kollicoat, solutol, ludipress and mixtures thereof.
lubricants such as magnesium stearate, stearic acid, sodium stearyl fumarate and mixtures thereof.
microcrystalline cellulose, crosslinked sodium carboxymethylcellulose, silica, Aerosil 200, corn starch, and mixtures thereof.
coatings.
a binding and gelling agent such as hydroxypropyl methocellulose (HPMC).
a colouring agent which may be a dye or a pigment. Suitable colouring agents are well known in the art and include curcumin, carotenoids, sunset yellow, tartrazine, indigo dyes, quino-phthalene dyes and triphenyl methane dyes.
antiflatulents such as simethicone, bulking agents such as polydextrose, antioxidants such as butylated hydroxyl toluene.
PEG-fatty acid esters with surfactant. The higher the molecular weight of the PEG used, the slower the formulation will dissolve. In addition, a molecular weight below 2500 is difficult to use in powder tablet equipment. Preferably, for a dry powder process producing a quick release formulation, the PEG molecular weight is between 3000 to 4000. Suitable PEG-fatty acid esters include those with a molecular weight up to 8000 and the fatty acid component can be selected from any suitable fatty acid such as laurate, dilaurate, oleate, stearate, glycerol trioleate, dioleate, glyceryl laurate, glyceryl oleate, palm kernel oil, hydrogenated castor oil, caster oil, corn oil, caprate/caprylate glycerides, polyglyceryl-10 laurate, phytosterols, cholesterol, soya sterol, sorbitan oleate and sorbitan laurate. Other examples of suitable PEGs include polysorbate 20, polysorbate 80, POE-9 lauryl ether, POE-23 lauryl ether, POE-10 oleyl ether, POE-20 oleyl ether, POE-20 stearyl ether, tocopheryl PEG-100 succinate, polyglyceryl-10 oleate, Tween 40, Tween 60, sucrose monostearate, sucrose monolaurate, sucrose monopalmitate, PEG 10-100 nonyl phenol series, PEG 15-100 octyl phenol series, a poloxamer, and mixtures thereof. The PEG can be selected to alter pharmacokinetics of the buccal matrix in a way to achieve either a zero or first order release rate depending upon the drug application. One skilled in the art of pharmaceutical drug delivery will appreciate that the selection of various alternative matrices will also alter the kinetics of the drug release across the buccal mucosa.

The selection of the PEG or PEG derivative and the amount used will depend on the active compound(s) to be included in the formulation. A person skilled in the art will be able to select a suitable PEG or PEG derivative to achieve the predetermined pharmacokinetics for a particular active ingredient because the properties of PEGs are well known. In particular, it has been known for some time that a low molecular weight PEG is usually a liquid whereas a higher molecular weight PEG tends to be a waxy solid.

It is also known that PEGs can complex with other compounds. Examples of such complexation include pegylation and PEG-fatty acid esters. These PEG complexes have different properties to the PEG alone which are useful when used in the present invention. For example, some pure uncomplexed PEGs having a molecular weight below 2000 floculate or exist as a liquid gel at room temperature which can make it difficult to use in a dry powder tabletting process. In contrast, the complexes of these low molecular weight PEGs are able to be used in a dry powder tabletting process. A person skilled in the art will know the properties of the different PEGs and PEG derivatives and be able to select the appropriate one to use with the selected active ingredient to provide the predetermined pharmacokinetics.

There are some doubts in the pharmaceutical industry regarding the use of PEG because of its associated carcinogenic potential due to trace contaminants. It is possible to use other excipients, such as chitosan and hyaluronic acid (which will deliver the same or a similar effect as PEG), should this be a concern.

Generally, the buccal and/or sublingual formulation according to the invention is capable of releasing the active compounds from within seconds to within hours and, more preferably, within at least about 60 minutes and, even more preferably, within about 40 minutes. Most preferably the buccal and/or sublingual formulation should be dissolved within 5 to 20 minutes but be capable of delivering drugs over an extended period.

The buccal and/or sublingual formulations of the present invention are expected to reduce the severity of gastrointestinal side-effects of particular active compounds. Symptoms of gastrointestinal irritation include indigestion, pain, nausea, vomiting, cramps, haemorrhaging, kidney damage, liver damage, diarrhoea and flatulence.

For example, the formulation according to the invention is expected to remove the need for the addition of esomeprazole, a potent proton pump inhibitor (PPI), added to some formulations to minimise the formation of gastric ulcers caused by the long-term use of NSAID for osteoarthritis patients.

The present invention further contemplates methods of treatment and/or prophylaxis of medical conditions in mammals and, in particular, humans by the administration of a drug delivery formulation which enhances the bioavailability of the drug, its salts or its metabolic derivatives, pro-drugs, intermediates or complexes. The expression “in need of” includes a subject directly requiring the formulation as well as situations where there is a perceived need to provide the formulation or where prophylaxis is required.

For example, there is a perceived need to develop a formulation having a prophylactic action to reduce the onset of Parkinson's disease. The Heart Research Institute is investigating using acetaminophen to inhibit the production of myeloperoxidase and the Harvard Medical School is investigating ibuprofen. Formulations according to the invention could be developed for these active compounds for use in these prophylactic treatments.

According to a further aspect of the invention there is provided a method for reducing the amount of compound necessary to achieve an effect in an individual as compared to a typical compound that is swallowed. The method comprises providing the buccal dosage forms of the present invention to an individual to achieve a specific effect. The buccal dosage form requires less than the typical amount of compound generally used in other formulations to achieve the effect. The buccal dosage form is placed in contact with the buccal membrane to thereby cause the compound to be released and absorbed optimally through the mucous membranes in a buccal cavity of the individual.

The formulation may be constructed in a manner known to those skilled in the art so as to give the predetermined controlled release of the compound. Typically, a formulation for a specific active compound will involve a multi step approach. By way of example, it may be that for a particular active compound, the issue of poor solubility (important for dissolution in the oral cavity) is addressed by pH adjustment or the addition of an enhancer or by altering the active compound by using its salt or some other derivative of the active compound. The same active compound might also exhibit poor membrane permeability and therefore require the addition of an enhancer to the formulation. It might also be possible to alter the structure of the active compound in different ways to facilitate its active transport across the buccal mucosa. Finally, the active compound, when released from the matrix, may exhibit an unacceptable taste. This would then require the inclusion of a suitable taste masking agent in the formulation. Where speed of onset is not considered a major factor, it may be viable to consider complexing the active compound, as an alternative to mask any taste, using a fatty acid or other compounds that may otherwise reduce membrane uptake of the bioactive compound or complex. A well known fact to one skilled in the art is that some complexation alternatives, while functioning effectively as taste maskers, also retard the uptake rate of the active.

In one embodiment, the buccal and/or sublingual delivery system is manufactured using a dry manufacturing process with all the components blended in a normal dry powder process and compressed using a standard tabletting machine. Such dry formulations can be manufactured in commercial numbers and provided in conventional blister packaging. This process is applicable where the excipients are chosen to eliminate the need for any wet formulation or semi manual processing which are costly and time intensive.

### DRAWINGS

Various embodiments/aspects of the invention will now be described with reference to the following drawings in which,

FIG. 1 shows the In Vitro Dissolution Data from Example 1.

FIG. 2 shows the Mean Concentration Time Profile data from Example 1.

FIG. 3 shows the Mean Dose Normalised (to 100 mg) Concentration-Time Profiles from Example 1.

FIG. 4 sets out the Pharmacokinetic Parameter Results from Example 1.

FIG. 5 sets out the Summary Pharmacokinetic Parameters from Example 1.

FIG. 6 shows the Dose Normalised Data from Example 3.

FIG. 7 shows the Dose Normalised AUC Values from Example 3.

FIG. 8 shows the ideal dose normalised curve for ibuprofen.

FIG. 9 shows the venlafaxine blood plasma levels obtained in the prior art.

FIG. 10 shows the expected blood plasma levels for the formulation from Example 4 compared with those of the prior art.

### EXAMPLES

Various embodiments/aspects of the invention will now be described with reference to the following non-limiting examples.

### Example 1

This example investigated the pharmacokinetics (Tₘₐₓ, Cₘₐₓ and AUC) of naproxen to determine the effect of certain variables on the plasma drug levels [1]. In particular, the pharmacokinetics of an orally ingested commercially available tablet form (Naprogesic® Bayer) containing 275 mg of naproxen sodium were compared with those of a compounded buccal matrix containing either 100 mg naproxen sodium or 100 mg naproxen. The trials were carried out on a total of 9 patients of various ages, weights and gender.

As the bioavailability of orally delivered naproxen is high [2], it was not anticipated that, in this case, there will be any major benefit in bioavailability seen from the use of a buccal system. However, buccal delivery may be capable of achieving the same bioavailability as oral delivery but with a lower loading dose of the active compound. In addition, by-passing the gastrointestinal tract will eliminate the classic gastrointestinal problems [1,3] associated with oral delivery and then first pass metabolism in the liver.

A second aim of the study was to compare the pharmacokinetics of a formulation containing a naproxen salt (i.e. sodium) as the active versus a similar formulation containing naproxen base as the active. There is a significant difference in solubility between the two forms of naproxen [4]. Figures quoted for naproxen base and naproxen sodium solubility in phosphate buffer are 6.8 mg/ml for naproxen base and 200 mg/ml for the sodium salt [5]. Such a large difference in solubility gives rise to the expectation of a difference in the pharmacokinetics for the two different forms.

### Method:

The study was an open label, pharmacokinetic investigation in small group (n=9) of subjects of mixed gender and age. The order of the study was not randomised. In each case, a single dosage form was studied with plasma concentrations of naproxen determined over a dosage interval. Following a 1 week minimum wash out period, the subject was administered the alternative dosage form and again the naproxen plasma concentration was monitored over a dosage interval.

### Selection of Study Population

Subjects were healthy men and women of variable age who all met the inclusion and exclusion criteria as defined below.

Inclusion Criteria:
In good health
Aged between 35 and 70 years old
Body mass index between 20-35
Capable of providing informed consent

### Exclusion Criteria:

Regular use of pain controlling medication or abuse of alcohol or any drugs
Medical problems that could affect pharmacokinetics

### Treatments:

Two dosage forms were used during the trial—oral and buccal. The buccal was made available in two forms one having the active present as the base and the other as the sodium salt.

Oral—Commercially available naproxen sodium was used. The selected tablet was a Naprogesic® tablet manufactured for Bayer Australia (equivalent compound in a swallow formulation). These tablets contained 275 mg Naproxen present in the tablet as the sodium salt.

Buccal—formulations according to the present invention were prepared as per the table below. The formulations contained the equivalent of 100 mg naproxen either present as the naproxen sodium salt or naproxen base. Solubility trials on the formulations showed that both formulations dissolved in 20 to 30 minutes.

| | % of the Total by weight | |
|---|---|---|
| Component | Naproxen base | Naproxen sodium |
| Magnesium Stearate | <2% | <2% |
| (Flowing agent) | | |
| Sorbitol (binder and | up to 42% | up to 42% |
| solubility enhancer) | | |
| PEG 4500 (release agent) | 15% | 15% |
| Lactose (binder) | 20% | 20% |
| Flavour (Blackcurrant powder) | <0.1% | <0.1% |
| Stevia (sweetener) | 0.4 | 1.6% |
| Sodium bicarbonate | 0.50% | 0.50% |
| (disintegrant accelerator | | |
| and masking agent) | | |
| Naproxen base (Active) | 20% | — |
| Naproxen sodium (Active) | — | 21.30% |

Sorbitol fulfilled different functional roles including as a binder, a solubility enhancer and it can mask some of the milder bitter tasting actives.
PEG 4500 was used to enable a dry powder process and the predetermined rate of release of the naproxen.
The stevia content was varied slightly reflecting the difference in taste bitterness between the base (0.4% Stevia) and the salt (much worse) which had 1.6% Stevia as a sweetener.
Sodium bicarbonate is another multi-function excipient which affects the rate of dissolution as well as being an effective taste masker.

Samples of both the commercial tablet and the compounded buccal matrix were assayed to confirm naproxen contents. All were within 3% of the target dose.

### Blood Analytical Methodology:

### Blood Sample Preparation

The following preparation procedure (based on established methodology) was used for the samples
Weigh tube and record weight
Mix tube
Centrifuge for 5 minutes at 3000 rpm
Remove 1 ml of plasma and place into centrifuge tube
Add 2 ml of acetonitrile and mix well
Centrifuge for 5 minutes at 3000 rpm
Extract 1 ml of clear supernatant for chromatographic analysis.

### Analytical Procedure

Chromatographic analysis was carried out using commercially available gradient High Performance Liquid Chromatography equipment. The analytical method was developed in house using modifications to published methods and then checked for linearity:
Linearity R2=1.0
Maximum percentile error=±1%

Standards prepared from pure USP naproxen base were used for comparison. Internal standards were not used, however the method of standard additions was used on 3 samples to confirm the calibration and ensure no interference from the background matrix.

### Trial Details

All subjects were requested to fast for eight hours prior to administration of the treatment then allowed to eat a normal breakfast 1 hour later.

### Each Subject Either:

placed a single buccal formulation according to the invention in the cheek cavity or under the tongue, leaving it undisturbed to disintegrate and release the active compound. Each subject recorded the time taken for complete dissolution of the buccal formulation; or
ingested a single Naprogesic® tablet with a minimum amount of water to aid swallowing.

After the treatments had been administered, the subjects were allowed to eat. The first meal occurred one hour after administration of the treatment. Around four and a half hours after application of the treatment all the subjects had a light lunch. Water, tea and coffee were taken during the seven-hour trial.

Blood samples were extracted from subjects over the seven-hour period following application of the selected dosage form. The blood was taken as individual extractions using normal blood collection tubes and according to standard blood collection protocols.

The tubes were mixed immediately after sampling and stored refrigerated in preparation for processing the next day. Subsequent repeat analysis confirmed that, once centrifuged and refrigerated, plasma samples were stable for at least five days.

### Data Analysis and Pharmacokinetic Parameter Estimations

Raw data was collected from the HPLC and processed via integration. Chromatogram peak areas were utilised for analysis.

The resulting figures were calculated in terms of ng naproxen sodium per millilitre of blood plasma. The vacuum gel tubes used to extract the blood are designed to draw the same volume each time. To confirm this, the tubes were weighed prior to centrifuging. The raw data was then subjected to Area under the Curve analysis. This analysis produces figures for
AUC;
Cₘₐₓ; and
Tₘₐₓ

The AUC should be calculated from zero to a time at which the concentration has returned to its regular levels. Also, when making comparisons, one should insure that all AUC's are calculated for the same time intervals.

To produce true comparative data, a mathematical procedure was used to extrapolate the collected data for several additional hours to give a total of twenty-four hours data. This extended data was then re-analysed to give AUC₂₄ figures for all subjects.

The procedure used to extrapolate the data utilised the quoted half-life of naproxen.

Simply, it was assumed that several hours post Tₘₐₓ the naproxen would decline essentially according to the half-life rule. So, from the final tested point (at around seven hours) the decline in the naproxen was theoretically calculated in line with regularly documented half-life of 12 hours.

The resulting extrapolated curves were in line with the actual test data.

### Pharmacokinetic Data

Naproxen was detectable in plasma samples from all subjects and was well within the detectable range of the test procedure.

Composite curves were constructed in order to collect all data together. This was achieved by generating an average figure for each time point within a group. These averaged time points were then used to generate a composite curve that could be used as a convenient visual comparison between the groups.

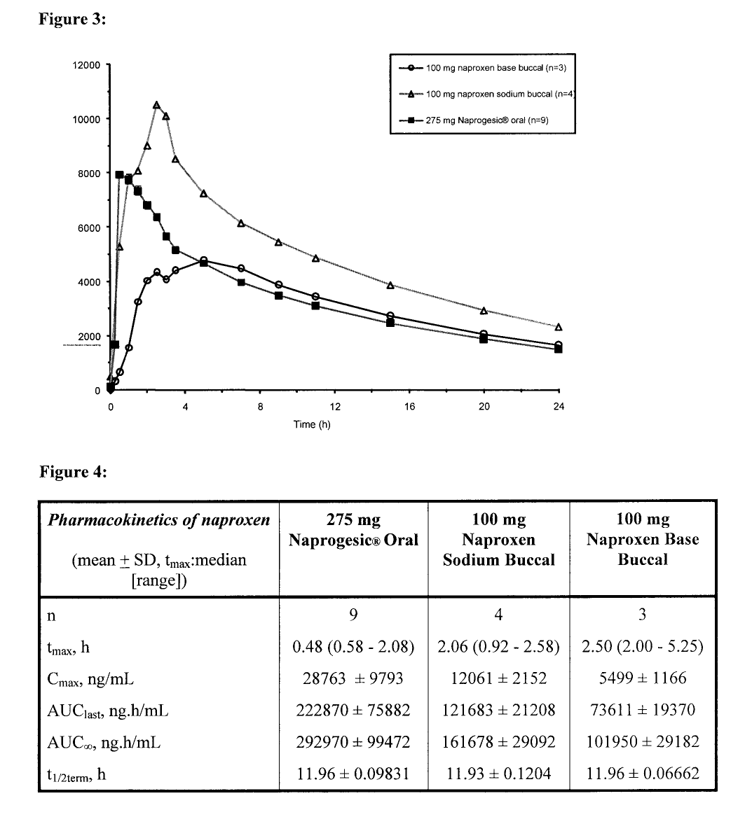

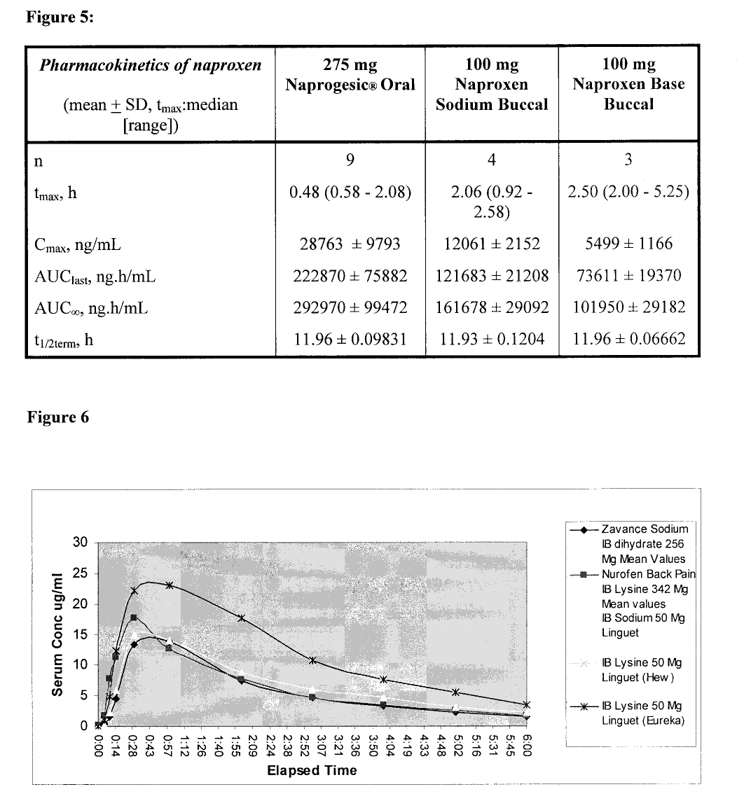

Previous work [14] indicated a near linear relationship between the applied dosage and the response in terms of Cₘₐₓ and AUC. By making this assumption it is reasonable that the response from the 275 mg tablet would be 2.75 times greater than that from the 100 mg buccal formulation according to the invention. To see this visually, an additional curve is included in the graph that shows the result of multiplying the 100 mg buccal formulation data by a constant factor of 2.75 (see FIG. 3).

### Results and Discussion

A number of previous studies have examined the pharmacokinetics of naproxen and its salt [6-15] and the ranges reported concur with the results obtained in this study.

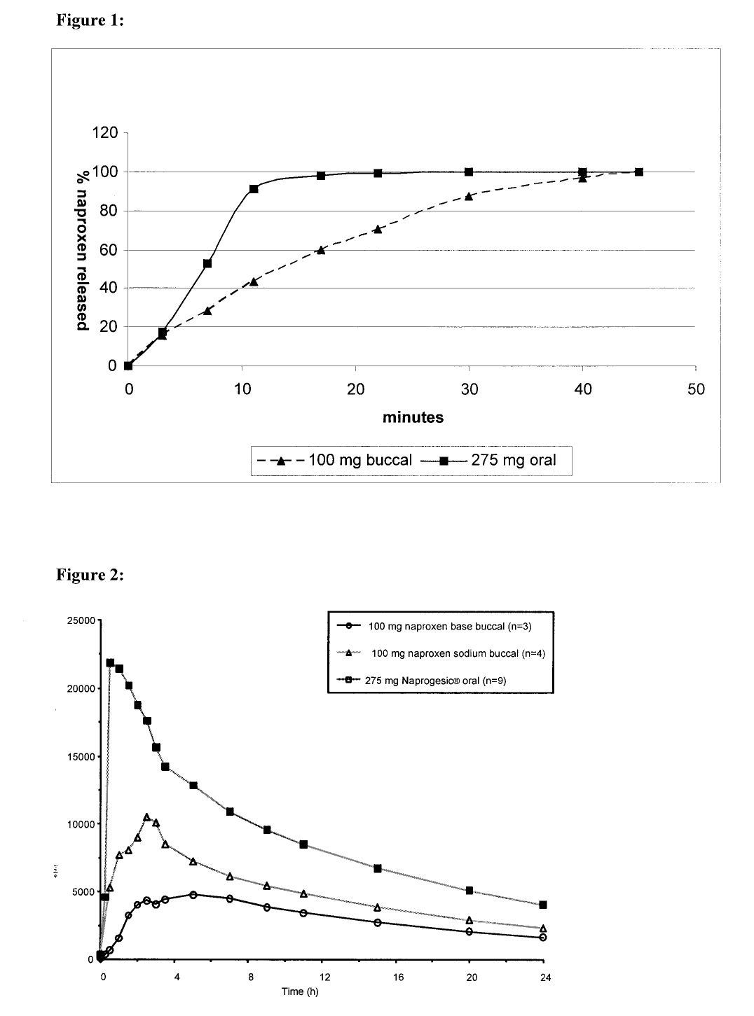

FIG. 1 illustrates simply that without any optimisation of the buccal formulation a sustained and controlled release was obtained, albeit slower in this case than the oral tablet equivalent. With subsequent optimisation of the formulation, it will be possible to shift the buccal formulation curve to the left producing a Tₘₐₓ at least equivalent to the tablet (in vivo).

FIG. 2 shows raw comparative data (serum blood levels) for 100 mg naproxen base, 100 mg naproxen sodium and 275 mg Naprogesic® tablet. In this unadjusted form, the indications are that onset is equivalent for both salt preparations which were also both superior to the naproxen base. As expected given the higher tablet dose, the AUC value is lower for the naproxen sodium buccal formulation. A Log graph of these results confirms that conclusion.

Compared to the unadjusted raw data, FIG. 3 shows a surprisingly very different picture. On a dose normalised basis, the naproxen sodium buccal formulation delivered the active just as quickly, but additionally produced a higher serum concentration of the active, than the commercially available Naprogesic® tablet. In addition, serum concentrations remained higher (AUC value) indicating potentially a superior pain relief outcome. This pain relief outcome was noted anecdotally by several trial subjects. The naproxen base exhibited lower bioavailability and was not taken up as quickly as expected given its poor solubility, but this should not be construed as eliminating naproxen base from consideration as a sustained pain relief product.

The dissolution profile indicates that an expected shift in Tₘₐₓ has been achieved in accordance with the invention. This further indicates significant and exciting potential to take optimisation further and improve the outcome given specific variant changes to the formulation. There is a higher maximum concentration and exposure (AUC) of naproxen sodium compared to naproxen base. On a dose normalised basis, the naproxen buccal formulation exhibited a higher Cₘₐₓ and AUC.

There were no reported adverse reactions from buccal administration of naproxen using this formulation and no significant indication of patient non-compliance (membrane irritation, throat catch or taste issues).

A significant majority of the anecdotal data suggested that patients found this new formulation to be a significant improvement over the original, and in some cases patients reported far longer pain relief window lasting up to 8 hours.

When using the formulation according to the invention, naproxen has been shown to be a suitable candidate for buccal administration having a bioavailability at least equal to if not superior to oral administration, with the advantage of bypassing the gastrointestinal tract and therefore avoiding all the associated side effects. Surprisingly, the results also suggest a higher serum response with a rapid onset of action (with equivalent dissolution) from a lower active dose compared to a three fold larger oral dose.

### REFERENCES

1. Martindale, The Complete Drug Reference, Pharmaceutical Press London, 35^{th Edition,} 2007 p 78.
2. Runkel R et al, J Pharm Sci., 61: (5) pp 703-708 (1972).
3. Place V, Darley P et al., Clin Pharmacol Theor vol 43, No 3, (March 1988)
4. Amaral M H, Lobo J M S et al. AAPS Pharm Sci/Tech, 2001 2(2) article 6.
5. Bhise K S et al, AAPS PharmSciTach; 8(2), Article 44 (2007).
6. Carmen Carrasco M, Herrera J E et al. Arznelm-Forsch/Drug Res. 56, No 8, 589-592 (2006).
7. Aarbakke J, Gadeholt G and Hoylanskjaer A, International Journal of Clinical Pharmacology Therapy and Toxicology, Vol 21, No 6, 281-283, (1983).
8. Desage J P et al., Journal of Clinical Pharmacology pp 189-193 (April 1976).
9. Vree T B et al, Biopharmacokinetics and Drug Disposition, Vol 14, pp 491-502 (1993).
10. Sastry M S P and Diwan P V., Arznelm-Forsch/Drug Res 43 (II) No 11 (1993).
11. Charles B G and Mogg G A G, Biopharmacokinetics and Drug Disposition Vol 13, pp 121-128 (1994).
12. Marzo A et al, Arznelm-Forsch/Drug Res 47 (I), pp 385-389, (1997)
13. Diansong Zhou et al, J Clin Pharmacol, 38, pp 625-629 (1998).
14. Niazi S K et al, Biopharmacokinetics and Drug Disposition, Vol 17, pp 355-361 (1996).
15. Strocchi et al, International Journal of Clinical Pharmacology, Therapy and Toxicology, Vol 29 No 7 pp 253-256, (1991).
16. Bourke D L, Smith T C. “Estimating allowable hemodilution”. Anesthesiology. 1974; 41: 609-612.
17. Hamman, Josias H. “Chitosan Based Polyelectrolyte Complexes as Potential Carrier Materials in Drug Delivery Systems” Mar. Drugs 2010, 8: 1305-1322.

### Example 2

Examples of formulations containing ibuprofen as the active compound according to the invention were prepared as follows (the proportions are all percentage by weight).

### Formulation 1

| | |
|---|---|
| Active | Ibuprofen lysine at 20% which is |
| | equivalent to 100 mg |
| Throat Catch agent | Carbomer 934P (971P or 974P) at 0.5-5% |
| | Miraculin at 2% |
| Flavour | Spearmint at 2% |
| Complexing | Hyaluronic acid at 20% |
| Agent/enhancer | |
| Permeation Enhancer | Lysalbinic acid 0.5% |
| Disintegrant and | Aluminium hydroxide at 1-2% and Sodium |
| masking agent | bicarbonate at 1% |
| Binder/Filler | Sorbitol at up to 42% but adjust to |
| | make up 100% (32-56%) |
| Flow Agent | Magnesium hydroxide at 2-5% |

### Formulation 2

| | |
|---|---|
| Active | Ibuprofen arginine at 20% which is |
| | equivalent to 100 mg |
| Throat Catch agent | Mixture of arginine with citric acid, |
| | oleic acid and glutamic acid at 1-10% |
| Flavour | Spearmint at 2% |
| Complexing Agent | PEG 3500 at 20% |
| Permeation Enhancer | Powdered ethanol (commercial product) |
| | at 0.5-1.0% |
| Disintegrant and | Sodium bicarbonate at least 1% |
| masking agent | |
| Binder/Filler | Erythritol at up to 42% but adjust to |
| | make up 100% (32-56%) |
| Flow Agent | Magnesium hydroxide or aluminium |
| | hydroxide at 5% |

### Formulation 3

| | |
|---|---|
| Active | Sodium ibuprofen dihydrate at 20% |
| | which is equivalent to 100 mg |
| Throat Catch agent | Carbomer 934P at 0.5-5% |
| Flavour | Spearmint at 2% |
| Release Agent | PEG 4000 at 25% |
| Permeation Enhancer | Sorbitol at 5% |
| Disintegrant and | Sodium bicarbonate at least 2% |
| masking agent | Mannitol at least 2% |
| Binder/Filler | Erythritol at up to 42% but adjust to |
| | make up 100% (32-56%) |
| Flow Agent | Magnesium stearate at up to 3% |

### Example 3

This example investigates the pharmacokinetic analysis of plasma ibuprofen concentration versus time profiles for different ibuprofen formulations.

### Methods

A clinical trial was conducted to obtain a results appropriate for statistical analysis. The methodology used in this Example was the same as that used in Example 1, except that there were 11 subjects.

### Treatments

1 Oral ibuprofen lysine (342 mg, equivalent to 200 mg ibuprofen; Nurofen® Back Pain). (equivalent compound in a swallow formulation)
2 Oral Sodium ibuprofen dihydrate (256 mg; equivalent to 200 mg ibuprofen; Nurofen® Zavance®). (equivalent compound in a swallow formulation)
3 Sublingual ibuprofen sodium Linguet™ formulation 50 mg (equivalent to 50 mg ibuprofen). This formulation was prepared according to the disclosure in WO 2006/105615.
4 Sublingual ibuprofen sodium Linguet™ formulation 100 mg (equivalent to 100 mg ibuprofen). This formulation was prepared according to the disclosure in WO 2006/105615.
5 Sublingual ibuprofen lysine Linguet™ Eureka formulation (equivalent to 50 mg ibuprofen). This formulation was prepared according to the invention.
6 Sublingual ibuprofen lysine Linguet™ Hewitt formulation (equivalent to 50 mg ibuprofen). This formulation was prepared according to the invention.

The sublingual formulations are described in more detail in the tables below.

Ibuprofen sodium Linguet ™ formulation 50 mg

| Excipient | Amount (mg) | % total |
|---|---|---|
| Ibuprofen sodium | 61.56 | 8.1 |
| Magnesium stearate | 15 | 2.0 |
| Sorbitol | 408 | 53.9 |
| Lactose | 150 | 19.8 |
| Stevia | 3.75 | 0.5 |
| PEG 3350 | 112.5 | 14.9 |
| Sodium bicarbonate | 3.75 | 0.5 |
| Citric Acid | 1.5 | 0.2 |
| Black currant | 1.5 | 0.2 |
| Total weight | 757.56 | 100.0 |

Ibuprofen sodium Linguet ™ formulation 100 mg

| Excipient | Amount (mg) | % total |
|---|---|---|
| Ibuprofen sodium | 123.12 | 27.8 |
| Carbomer | 9 | 2.0 |
| Lecithin | 36 | 8.1 |
| Spearmint | 9 | 2.0 |
| Stevia | 6 | 1.4 |
| PEG 3350 | 60 | 13.6 |
| Ethanol powder | 3 | 0.7 |
| Methyl cellulose | 22 | 5.0 |
| Sodium bicarbonate | 3 | 0.7 |
| Erythritol | 150 | 33.9 |
| Magnesium hydroxide | 6 | 1.4 |
| Aluminum hydroxide | 15 | 3.4 |
| Total weight | 442.12 | 100.0 |

Ibuprofen lysine Linguet ™ Eureka formulation

| Excipient | | Amount (mg) | % total |
|---|---|---|---|
| Ibuprofen lysine | | 85.5 | 22.0 |
| Carbomer | Throat catch | 9 | 2.3 |
| Lecithin | agents | 36 | 9.3 |
| Spearmint | Tastemasking | 9 | 2.3 |
| Stevia | agents | 6 | 1.5 |
| PEG 3350 | | 60 | 15.4 |
| Ethanol powder | | 3 | 0.8 |
| (permeation enhancer) | | | |
| Methyl cellulose | | 22 | 5.7 |
| Sodium bicarbonate | | 3 | 0.8 |
| Erythritol | | 140 | 36.0 |
| Magnesium hydroxide | Buffering | 7.5 | 1.9 |
| Aluminum hydroxide | agents | 7.5 | 1.9 |
| Total weight | | 388.5 | 100.0 |

Ibuprofen lysine Linguet ™ Hewitt formulation

| Excipient | Amount (mg) | % total |
|---|---|---|
| Ibuprofen lysine | 85.5 | 10.9 |
| Magnesium stearate | 15 | 1.9 |
| Sorbitol | 408 | 52.2 |
| (permeation enhancer) | | |
| Lactose | 150 | 19.2 |
| Stevia (taste | 3.75 | 0.5 |
| masking agent) | | |
| PEG 3350 | 112.5 | 14.4 |
| Sodium bicarbonate | 3.75 | 0.5 |
| Citric acid | 1.5 | 0.2 |
| Blackcurrant | 1.5 | 0.2 |
| Total weight | 781.5 | 100.0 |

The individual and group mean data was transferred into WinNonLin Pro Node 5.2™ and subjected to pharmacokinetic analysis. The following pharmacokinetic parameters were calculated for the individual and group mean data: area under the curve (AUC); terminal phase elimination rate constant (λz); maximum concentration (Cₘₐₓ); time to reach maximum concentration (Tₘₐₓ); and terminal half life (T_{1/2}). Pharmacokinetic parameters were calculated using a noncompartmental analysis (NCA) model. A uniform weighting scheme was used for the determination of the elimination rate constant and half-life. AUC values for the plasma ibuprofen concentration profiles were calculated using the linear trapezoidal rule up to the last measurable sampling time point (AUC₀₋ₗₐₛₜ) and extrapolated to infinity (AUC_{(0-inf)}).

The following parameters were then calculated for the mean dose normalised values.
AUC_{(O-inf)} Area Under Curve extrapolated to infinity.
Cₘₐₓ (ng/ml) Maximum concentration
Tₘₐₓ (hr) Time to reach maximum concentration

### Results

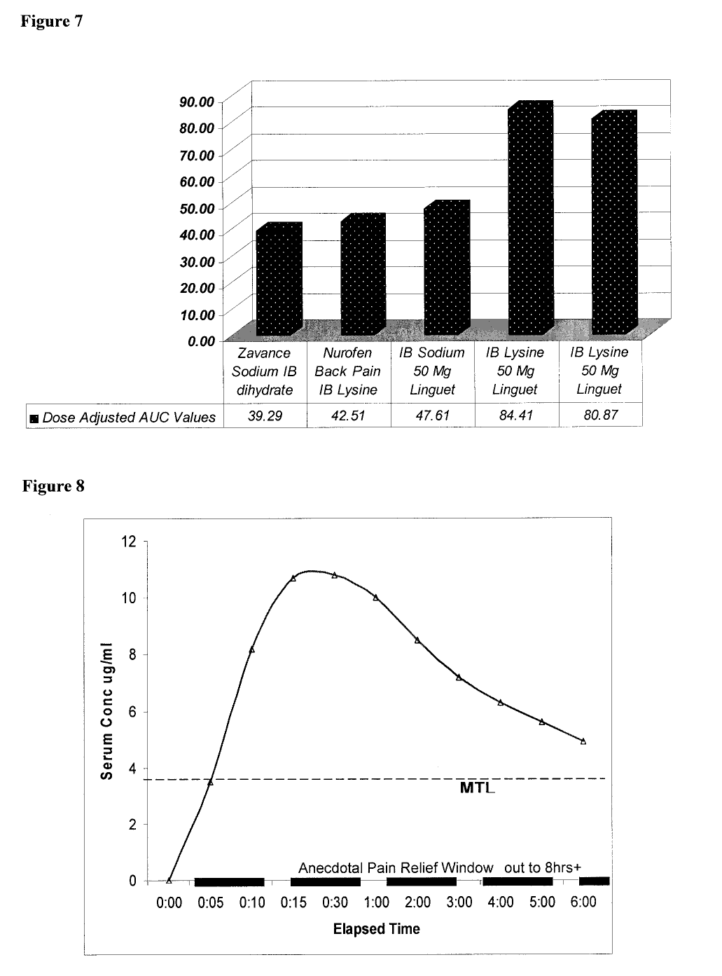

The ibuprofen concentration versus time profiles for the differing formulations show similar kinetics with a fast increase in concentration up to a maximum and then a relatively slower decrease in concentration over time. The most marked differences between the formulations are observed in the AUC values. FIGS. 6 and 7 provide a visual representation of the Cₘₐₓ and AUC results.

| | Zavance ® | Nurofen ® | IB | IB Lysine | IB Lysine |
|---|---|---|---|---|---|
| Mean Dose | Sodium IB | Back Pain | Sodium | 50 Mg | 50 Mg |
| Normalised | dihydrate | IB Lysine | 50 Mg | Linguet ™ | Linguet ™ |
| Values | 256 Mg | 342 Mg | Linguet ™ | (Hew) | (Eureka) |
| Cₘₐₓ | 13.80 | 17.75 | 15.00 | 26.80 | 23.10 |
| Tₘₐₓ | 1.00 | 0.50 | 1.00 | 0.50 | 1.00 |
| AUC(0-inf) | 39.29 | 42.51 | 47.61 | 84.41 | 80.87 |

### CONCLUSION

FIGS. 6 and 7 clearly illustrate that the two ibuprofen lysine formulations according to the invention had significantly better pharmacokinetics than either of the formulations according to WO 2006/105615 or the current oral formulations (Nurofen® Back Pain and Zavance®). Further, these improved pharmacokinetics are with respect to an earlier onset of action and release over an extended period of time. In addition, these results were achieved using a lower dose and were in line with the optimised graphical representation as depicted in FIG. 8 (ie a predetermined release rate).

In addition to the improved control over the pharmacokinetics provided by complexing agents and membrane permeability enhancers, the use of taste masking agents (which deal with throat catch, buffering and flavour) was reported by trial subjects to significantly improve mouthfeel and virtually eliminate throat catch and taste issues. These qualitative elements emerge as significant commercial drivers when patient compliance with any formulation to be taken into production is considered.

### Example 4

In this example, a formulation is developed according to the invention for venlafaxine hydrochloride (an antidepressant).

| Excipient | Amount (mg) | % total |
|---|---|---|
| Venlafaxine hydrochloride | 75 | 12.0 |
| (equivalent to 75 mg) | | |
| Carbomer | 10 | 1.6 |
| Benecoat ™ | 40 | 6.4 |
| Coffee/vanilla extract | 10 | 1.6 |
| Stevia | 8 | 1.3 |
| PEG 3350 | 90 | 14.4 |
| Ethanol powder | 4.5 | 0.7 |
| Methyl cellulose | 30 | 4.8 |
| Sodium bicarbonate | 4 | 0.6 |
| Erythritol | 140 | 22.3 |
| Sorbitol | 200 | 31.9 |
| Magnesium hydroxide | 7.5 | 1.2 |
| Aluminium hydroxide | 7.5 | 1.2 |
| Total weight | 626.5 | 100.0 |

The aim of this formulation is to provide a faster speed of onset with an equivalent or slightly lower Cₘₐₓ but with a significantly higher AUC value or therapeutic treatment window than the extended release formulation disclosed in AU2003259586 (equivalent compound in a swallow formulation). AU2003259586 has been used as a commercial reference and as a basic indicator of what optimisation potential should be expected through using this invention.

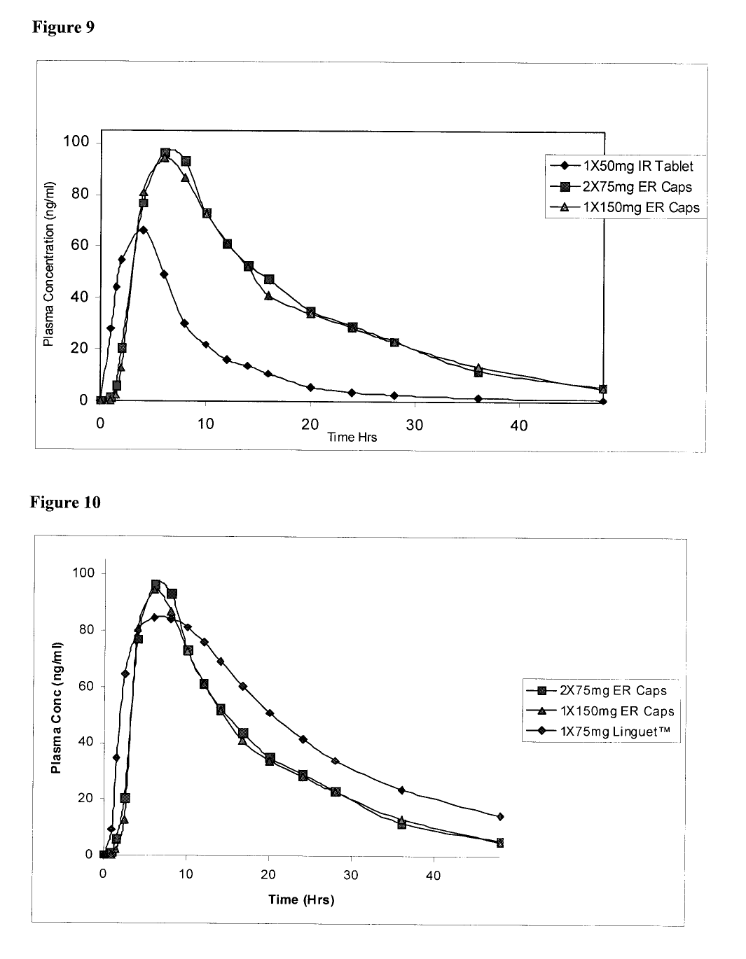

The superior AUC will be evidenced by a longer “tail” on the plasma concentration vs. time curve. FIG. 9 depicts a graphical representations of the results from AU2003259586 and FIG. 10 illustrates what is expected to be achieved using a formulation according to the invention (ie a predetermined release rate).

FIG. 10 indicates that an expectation of delivering a superior outcome off a significantly lower dose (75 mg once daily) is possible using an optimal variant of the above formulation. The implications for patient compliance (once daily dose with no side-effects) are very positive.

### Example 5

This is a further example of a formulation according to the invention containing melatonin as the active compound.

| Excipient | Amount (mg) | % total |
|---|---|---|
| Melatonin* | 2.5 | 2.8 |
| Magnesium stearate | 1.3 | 1.4 |
| Sorbitol | 65 | 72.5 |
| Stevia | 2.5 | 2.8 |
| PGA Base B | 10.5 | 11.7 |
| Ethanol powder | 3 | 3.3 |
| Citric acid | 0.4 | 0.4 |
| Sodium bicarbonate | 0.5 | 0.6 |
| Plasdone S630 | 4 | 4.5 |
| Total weight | 89.7 | 100.0 |

*note
equivalent to 10.2 mg theoretically active concentration

This formulation has been prepared in several preliminary batches used to confirm tabletting procedures, release rates and dissolution times. This formulation has a dissolution time of 24 minutes measured using a standard dissolving test (roller method, using a belt roller apparatus).

Although no plasma concentration studies have been completed with this formulation, the inventors anticipate that a similar result (Linguet™ vs. oral dose) as those shown in the examples above will be achieved. That is, a superior treatment window (higher AUC dose normalised value) generated using a lower dose with a correspondingly more patient compliant safety profile.

### Example 6

Formulations according to the invention containing sterolin as the active compound were prepared.

### Formulation 1

| Excipient | Amount (mg) | % total |
|---|---|---|
| Sterolin* | 5 | 10.0 |
| Magnesium stearate | 1 | 2.0 |
| Sorbitol | 37.5 | 75.0 |
| PGA Base B | 4.5 | 9.0 |
| Plasdone S630 | 2 | 4.0 |
| Total Weight | 50 | 100.0 |

*note
equivalent to a theoretical active concentration of 22.0 mg

This formulation has a dissolution time of 48 minutes measured using a standard dissolving test (roller method, using a belt roller apparatus).

### Formulation 2

| Excipient | Amount (mg) | % total |
|---|---|---|
| Sterolin* | 2.5 | 5.0 |
| Magnesium stearate | 1 | 2.0 |
| Sorbitol | 35 | 70.0 |
| PGA Base B | 9 | 18.0 |
| Citric acid | 0.25 | 0.5 |
| Plasdone S630 | 2 | 4.0 |
| Sodium bicarbonate | 0.25 | 0.5 |
| Total Weight | 50 | 100.0 |

*note
equivalent to a theoretical active concentration of 22.5 mg with half the dissolution time

This formulation has a dissolution time of 24 minutes measured using a standard dissolving test (roller method, using a belt roller apparatus).

### Example 7

A formulation according to the invention containing ibuprofen lysine in combination with cetirizine (antihistamine) as the active compounds was prepared.

Ibuprofen lysine/cetirizine Linguet ™ formulation

| Excipient | | Amount (mg) | % total |
|---|---|---|---|
| Ibuprofen lysine | | 85.5 | 22 |
| Cetirizine | | 1 | 2 |
| Carbomer | Throat catch | 9 | 2.4 |
| Lecithin | agents | 36 | 9.3 |
| Spearmint | Taste masking | 9 | 2.3 |
| Stevia | agents | 6 | 1.5 |
| PEG 3350 | | 60 | 15.4 |
| Ethanol powder | | 3 | 0.8 |
| (permeation enhancer) | | | |
| Methyl cellulose | | 22 | 5.7 |
| Sodium bicarbonate | | 3 | 0.8 |
| Erythritol | | 140 | 36.0 |
| Magnesium hydroxide | Buffering | 7.5 | 1.9 |
| Aluminum hydroxide | agents | 7.5 | 1.9 |
| Total weight | | 389.5 | 100.0 |

The projected dissolution time for this formulation is 15-20 minutes to be measured using a standard dissolving test (roller method, using belt roller apparatus).

### Example 8

A formulation according to the invention containing glucosamine as the active compound was prepared.

| Excipient | Amount (mg) | % total |
|---|---|---|
| Glucosamine | 265.2 | 51 |
| Magnesium stearate | 7.8 | 1.5 |
| Sorbitol | 119.6 | 23 |
| Hyaluronic acid | 57.2 | 11 |
| Caramel flavour | 2.6 | 0.5 |
| Plasdone S630 | 26 | 5 |
| Ethanol powder | 41.6 | 8 |
| Total weight | 520 | 100.0 |

A 520 mg Linguet™ will deliver a theoretically active conc. around 260 mg This formulation has a dissolution time of 29 minutes measured using a standard dissolving test (roller method, using a belt roller apparatus).

The word ‘comprising’ and forms of the word ‘comprising’ as used in this description and in the claims does not limit the invention claimed to exclude any variants or additions.

Modifications and improvements to the invention will be readily apparent to those skilled in the art. Such modifications and improvements are intended to be within the scope of this invention.

The claims defining the invention are as follows:

1. A buccal and/or sublingual formulation comprising:
(a) one or more active compounds; and
(b) a matrix which releases the active compounds at a predetermined rate for transport across the buccal and/or sublingual membranes, the matrix comprising one or more compounds selected predominantly from the group consisting of:
(i) taste masking agents,
(ii) enhancers,
(iii) complexing agents, and
mixtures thereof; and
(c) other pharmaceutically acceptable carriers and/or excipients,
wherein the rate of release of the active compounds is either (A) the same or substantially the same rate at which the active compounds are transported across the buccal and/or sublingual membranes; or (B) a rate which releases the active compounds so as to provide a higher area under the curve (AUC) value when compared with equivalent compounds in a swallow formulation on a dose normalised basis.

2. A buccal and/or sublingual formulation according to claim 1 wherein the matrix comprises one or more compounds selected from the group consisting of PEGs, chitosan, hyaluronic acid, cyclodextrins, polyalcohols, and mixtures thereof.

3. A buccal and/or sublingual formulation according to claim 1 wherein:
(a) the one or more active compounds comprises one or more compounds selected from the group consisting of naproxen, ibuprofen, venlafaxine, glucosamine, cetirizine, melatonin, sterolin, pharmaceutically acceptable salts thereof, pharmaceutically acceptable derivatives thereof and mixtures thereof;
(b) the matrix comprises one or more compounds selected from the group consisting of carbomer, lecithin, sodium bicarbonate, spearmint, stevia, sorbitol, mannitol, caramel flavour, Plasdone S630, ethanol powder, magnesium hydroxide, aluminium hydroxide, citric acid, Miraculin, hyaluronic acid, lysalbinic acid, PGA Base B, blackcurrant powder and mixtures thereof and
(c) the other pharmaceutically acceptable carriers and/or excipients comprises one or more compounds selected from the group consisting of PEGs 3300 to 4500, magnesium stearate, erythritol, lactose, methyl cellulose and mixtures thereof.

4. A method for reducing the amount of active compound used to achieve an effect in an individual patient as compared to a typical compound that is swallowed, the method comprising administering to an individual a buccal and/or sublingual formulation comprising
(a) one or more active compounds; and
(b) a matrix which releases the active compounds at a predetermined rate for transport across the buccal and/or sublingual membranes, the matrix comprising one or more compounds selected predominantly from the group consisting of:
(i) taste masking agents,
(ii) enhancers,
(iii) complexing agents, and
mixtures thereof; and
(c) other pharmaceutically acceptable carriers and/or excipients,
wherein the rate of release of the active compounds is either (A) the same or substantially the same rate at which the active compounds are transported across the buccal and/or sublingual membranes; or (B) a rate which releases the active compounds so as to provide a higher area under the curve (AUC) value when compared with equivalent compounds in a swallow formulation on a dose normalised basis.
